(19) United States
(12) Patent Application Publication
(10) Pub. No.: US 2022/0287698 A1
(43) Pub. Date: Sep. 15, 2022

(54) PERCUTANEOUS WOUND BARRIER

(71) Applicant: ST. CROIX SURGICAL SYSTEMS, LLC, Lakeland, MN (US)

(72) Inventor: Karol Leon Nowakowski, Lakeland, MN (US)

(21) Appl. No.: 17/750,985

(22) Filed: May 23, 2022

Related U.S. Application Data
(63) Continuation-in-part of application No. 17/197,845, filed on Mar. 10, 2021.

(51) Int. Cl. A61B 17/00; A61B 17/34

(52) U.S. Cl. CPC A61B 17/0057; A61B 17/3421; A61B 17/00491; A61B 2017/00654; A61B 2017/00884

(57) ABSTRACT

A surface-modified cannula includes a hollow shaft having a proximal opening and one or more surface features along a portion of the length of the hollow shaft, the one or more surface features including one or more channels, one or more depressions, and/or two or more ports each extending at least partially between an outer diameter and an inner diameter of the hollow shaft. The one or more surface features are configured to enable, upon delivery of the cannula to a position proximate to a wound site in the blood vessel, collection of the patient's blood from the wound site, and dispersal of the blood along an access path to the wound site, thereby enabling blood to migrate from the wound site to a region surrounding and extending from the wound site along the access path.

### CROSS-REFERENCE TO RELATED APPLICATIONS

This application claims priority to and is a Continuation-in-Part of U.S. patent application Ser. No. 17/197,845, filed Mar. 10, 2021. This application is related to U.S. Pat. Nos. 10,973,503 and 10,231,721. All above identified applications are hereby incorporated by reference in their entireties.

### BACKGROUND

Blood vessels are commonly used as a conduit to access internal patient anatomy for assessing medical needs and performing surgical procedures. Access through blood vessels allows surgical procedures to be performed while greatly reducing trauma and recovery time for the patient. Such procedures are generally regarded as minimally invasive procedures in contrast to open surgery procedures, the latter of which cut a patient open for access and create much larger wounds to be closed following a surgical procedure.

In 1953, Sven Seldinger developed a minimally invasive percutaneous access technique that is still commonly used today. This technique, known as Seldinger access, typically consists of several basic steps. A blood vessel, such as the femoral artery, is punctured through the skin surface using a hollow syringe needle. A guidewire is threaded through the needle into the artery, and the needle is removed by sliding it out over the guidewire. A cannula known as a dilator is inserted through a larger diameter tube known as a sheath, and both are advanced over the guidewire into the blood vessel, thus also assisting with later closure of the wound by having minimized disruption of the wounded tissue. The dilator and guidewire are removed from the sheath, leaving the sheath spanning from the outside of the patient to the inside of the blood vessel. The sheath provides an access port to the inside of the blood vessel through which large-diameter catheters and other surgical instrumentation may be advanced into, and traversed around, the patient's body. The sheath also serves to seal the wound from bleeding prior to completion of a further medical procedure through the wound. An anticoagulant such as heparin is typically administered to the patient so that the instrumentation placed into the blood vessel does not precipitate dangerous blood clots within the vasculature. Upon completion of the medical procedure, all instrumentation and the sheath are removed from the patient. The access wound site is typically subjected to manual compression until a clot has established to sufficiently stop bleeding from the vessel wound.

The use of manual compression for wound closure following a minimally invasive percutaneous surgical procedure is the “gold standard” by which all alternative methods of wound closure are evaluated for safety, reliability, and efficacy. However, wound closure by manual compression can be a slow process, particularly in the presence of anticoagulants. This led to a plethora of wound closure inventions that sought to expedite wound closure. Some, by example Khosravi et al. in US Application 2005/0149117 A1, followed Seldinger's efforts to seal or assist sealing of percutaneous wounds early in the process of performing a percutaneous medical access procedure. Khosravi did this by early deployment of a wound closure approach selected from a broad choice of agents, materials, or devices to seal or assist in sealing a percutaneous wound site. Despite the above prior art and numerous other percutaneous wound closing inventions, acceptable success rates remain elusive in the clinic due largely to external reasons, e.g., inherent anatomical variability in patients, with the resultant persistent bleeding complications regarded as far outweighing the cardiovascular complications of the primary reason for a surgical procedure. This is summarized in an article authored by Jackson Thatcher, MD, FACC, Director of Inpatient Cardiology for the Park Nicollet Heart Center at Methodist Hospital St. Louis Park, for Cath Lab Digest, (March 2008,) entitled “Groin Bleeds and other Hemorrhagic Complications of Cardiac Catheterization: A list of relevant issues” Volume 16 (Issue 3).

The article lists percutaneous accessed arterial bleeds as the number one “ . . . major cause of morbidity and mortality associated with cardiac catheterization procedures and percutaneous coronary interventions” with failed percutaneous wound closure technologies alone including only a very small portion of the root causes cited. In contrast, the embodiments described in the present disclosure compensate for the vast majority of root causes cited in the article, not by offering yet another percutaneous wound closure technology, but by providing a barrier to protect a patient when a wound closure technology fails or is otherwise rendered ineffective in the presence of contributing factors.

### SUMMARY OF CERTAIN EMBODIMENTS OF THE DISCLOSURE

In one aspect, the present disclosure provides for various systems, methods, and devices to reduce or prevent percutaneous wound bleeding complications.

In one aspect, the present disclosure provides for a failsafe barrier to internal bleeding pathways when a percutaneous wound closure fails to stop bleeding.

In some embodiments, methods and apparatus described herein may be used to direct vessel bleeding out a wound site access tract of the patient and away from other subcutaneous anatomical areas of a patient.

The methods and apparatus described herein, in some embodiments, may be used in combination with other wound closure systems, methods, and devices, to assist in or enhance closing a wound while also serving to substantially block internal bleeding pathways leading to complications.

The methods and apparatus described herein, in some embodiments, may be used to alter the anatomical structure of a percutaneous wound area through creation of substantial blood tissue capable of isolating internal bleeding pathways from the source of bleeding.

The methods and apparatus described herein, in some embodiments, may be supplied with an indicator to allow control of pressure buildup when filling a failsafe barrier mold cavity.

The methods and apparatus described herein, in some embodiments, may be combined with enhancements including management of anticoagulants, clot initiators, clot accelerants, pain killers, anti-lytic agents, and the like.

The methods and apparatus described herein, in some embodiments, may be supplied as a kit by itself or in combination with components used for any other procedure to be performed upon a patient.

The objects and advantages will appear more fully from the following detailed description in conjunction with the accompanying drawings.

### BRIEF DESCRIPTION OF THE DRAWINGS

FIG. 1 depicts a side elevation view of a collection of exemplary elements for creating a percutaneous wound bather.

FIG. 2 depicts a side elevation partial cutaway view of elements involved in one step of a preferred approach as described herein, where the sheath accesses a vessel by extending into the vessel through the skin and typically loose connective tissue surrounding vessel, such as through the Seldinger technique.

FIG. 3 depicts a side elevation partial cutaway view of elements involved in certain embodiments of creating a percutaneous wound barrier, where the catheter is attached to the syringe and advanced down a tissue tract alongside the sheath without advancing into the vessel.

FIG. 4 depicts a side elevation partial cutaway view of elements involved in further embodiments of creating a percutaneous wound barrier, where blood is injected from the syringe through the catheter to around the vessel, vessel wound (now hidden), and along the tissue tract around the sheath until the blood preferably emerges at the skin surface access site.

FIG. 5 depicts a side elevation partial cutaway view of elements involved in certain embodiments of creating a percutaneous wound barrier, where the syringe and catheter are removed from the patient while the blood typically continues clotting, and the sheath is available for intravascular medical procedures.

FIG. 6 depicts a side elevation partial cutaway view of preferred elements of the clotted failsafe barrier upon removal of instruments from the patient's wound site, including the typically clotted blood tissue casting both encasing the vessel, its wound (not shown) and extending through the tissue tract to the skin surface access site.

FIG. 7 depicts a radial elevation partial cutaway view of preferred elements of the clotted failsafe barrier upon removal of instruments from the patient's wound site, including the typically clotted blood tissue casting substantially encasing the vessel with its wound, and extending through the tissue tract to the skin, and typically having an open channel impression molded within the blood tissue casting left behind by removed instrumentation (not shown), thus preferably directing any bleeding from the wound along the channel, to out of the patient's skin at the access site and substantially preventing internal bleeding pathways into areas of the typically surrounding the tissue and beyond (not shown).

FIGS. 8A and 8B illustrate example surface-modified needles or cannulas.

FIGS. 9A through 9G illustrate another example surface-modified needle or cannula.

FIG. 10 is a flow chart of an example method for using a surface-modified needle or cannula.

### DETAILED DESCRIPTION OF THE PREFERRED EMBODIMENTS

One aspect of the approaches described herein includes using familiar elements of a percutaneous medical procedure for new functions. These functions may also be described in the context of method steps, systems or system components, apparatus, or any combination thereof for creating a percutaneous wound barrier.

When referring to bleeding “complications” or “complication” and the like the general intent, unless expressly stated otherwise, is to refer to blood causing unacceptable blood accumulation and/or transfer beneath the skin such as in pseudoaneurysms, hematomas, retroperitoneal bleeding, and the like.

When referring to a “vessel” and absent any stipulation to the contrary, this generally refers to the vessel subject of the arteriotomy for vascular access.

The teachings herein depart from a conventional way of looking at its elements. For example, the subcutaneous area including interstitial loose connective tissue, blood vessels, cutaneous tissue, muscle, and like anatomical features in the access site area may be referred to as a “mold” or the like with an introducer sheath or like instrument, when present, representing what may be referred to as a “core pin” extending into the mold. Clotting material, such as blood, injected to fill the mold subsequently solidifies to become what may be referred to as a mold “casting.” The casting need not be removed from the mold, and typically both the casting and parts of the mold, like the tissue tract, would be reabsorbed or the like by the patient's body over time. Concerning vessel wound management in general, the teachings herein are largely unconcerned with directly the vessel's wound puncture or its closure and are more concerned with the surrounding anatomical space and any inserted instruments. The preferred intent here is to form a failsafe barrier substantially encasing the entire wound area and vessel to preventing bleeding from a failed wound closure, generally accomplished by preferably filling and clotting within internal anatomical spaces and fluid pathways that could otherwise give rise to bleeding complications when a wound closure fails. Users wanting to provide the patient such a failsafe barrier may also find benefits in a subsequently applied wound closure's performance including cost savings, improved ease of use, improved success rate, ability to use with larger French size instruments and vessel holes, or the like. As such, one embodiment also specifically includes providing a failsafe bather to internal bleeding pathways and enhanced performance of a wound closure approach used.

The clotting or clottable agent may be anything that converts from a material that can flow, into a material that is substantially stationary with liquids, gels, beads, and powders including four such examples. In the case of using blood, the clotted blood can also be considered to form a blood “tissue” when clotted. In such case, the methods described herein can also be thought of as changing the patient's anatomy that is subject to a subsequent wound closure.

Performance enhancements that may be used with some embodiments for initiating or accelerating clot formation, reducing lysis of formed clot, providing pain reduction, providing clotting agent radiopacity to observe placement, and the like are discussed in the reference documents and other publications. Likewise, the choice of clotting agents, sequence of deploying a clotting agent or agents, timing of deploying a clotting agent or agents, options for apparatus, methods, and systems employed to practice the methods can all be selected from the reference documents and other publications.

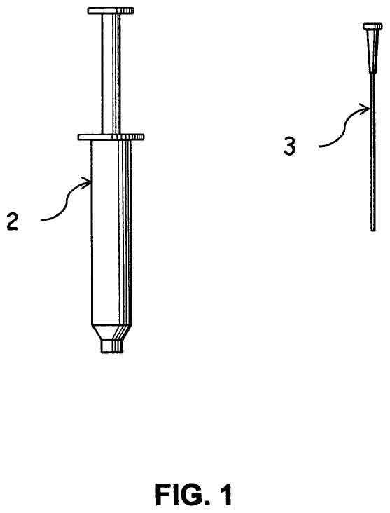

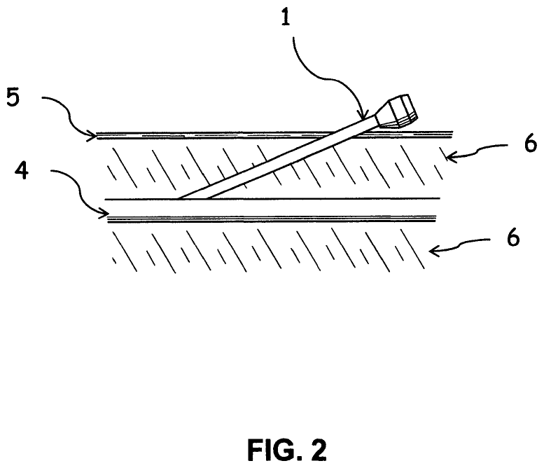

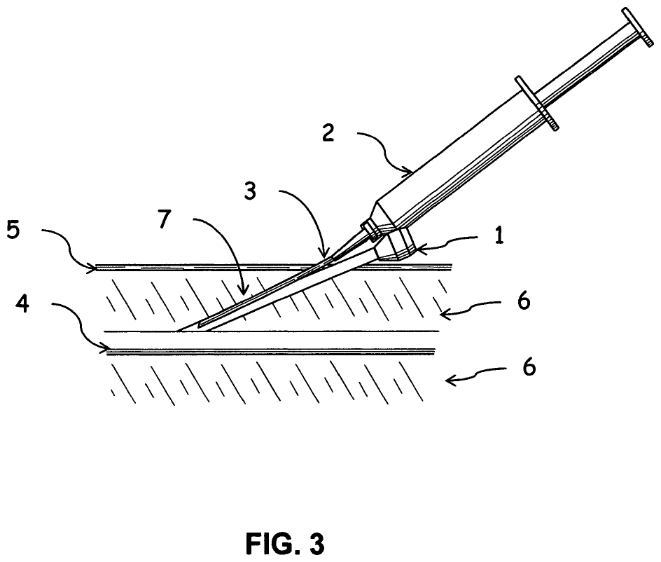

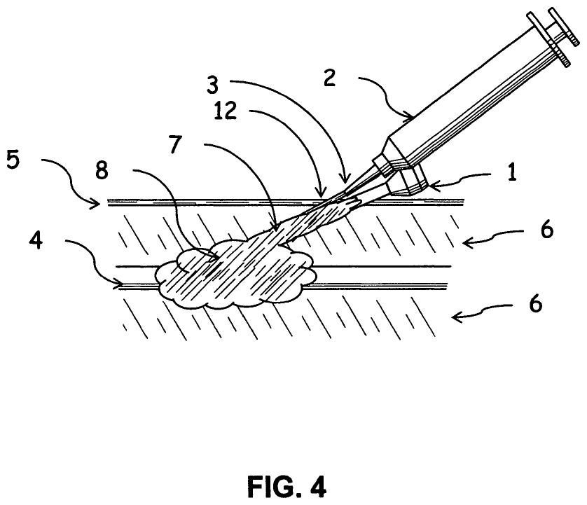

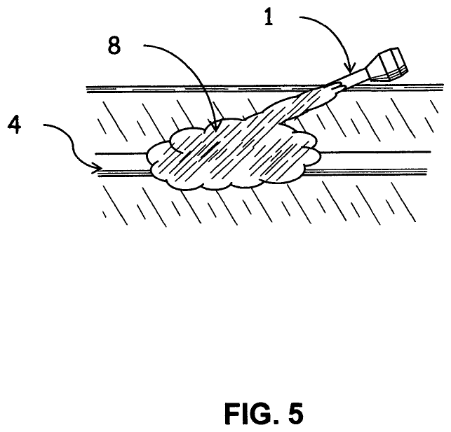

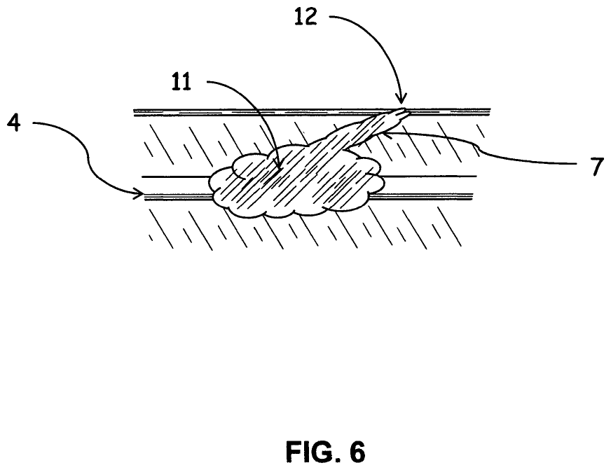

A preferred embodiment begins with apparatus illustrated in FIG. 1 typically including a disposable syringe 2 and cannula 3, exemplifying both the potential very low cost and simplicity benefits. The cannula may include any instrument for providing fluid communication between a source of clottable agent and the mold cavity. In FIG. 2, an instrument such as an introducer sheath 1 is inserted through the skin 5, other tissue 6 generally surrounding the vessel 4, and into the vessel 4, as is typically done to establish a percutaneous access site for a medical procedure. The syringe 2 of FIG. 1 is typically filled with blood available through the sheath 1 from vessel 4 of FIG. 2. The blood-filled syringe 2 and cannula 3 of FIG. 1 are combined, and as shown in FIG. 3, then inserted through the skin 5 alongside the sheath 1 through tissue tract 7. Referring to FIG. 4, a sufficient volume of clottable blood 8 to create a failsafe barrier, is injected to fill the general wound site area thereby typically encasing both vessel 4 and sheath 1 as present in tissue tract 7 until injected blood typically flows out of the skin 5 access site 12. Absent an optional pressure gauge, the user can watch for unacceptable swelling at the skin surface, and if needed, also use the syringe to remove some of the injected blood to reduce pressure within the mold cavity and internal bleeding pathways when present through or around other tissue 6. In FIG. 5, the deposited blood advances its clot formation toward becoming a solid tissue mass while sheath 1 remains available for use in other intravascular medical procedures. In FIG. 6, the access sheath 1 (not shown) has been removed and the clotting blood has transformed into the substantially solid tissue mass casting 11. In cross section illustration of FIG. 7, details of the substantially solid tissue mass casting 11 are illustrated showing it substantially encasing vessel 4, vessel wound 10, and extending through the tissue tract 7 to the skin 5, and typically having a core pin channel impression 9 molded within as left behind by removed instrumentation (not shown,) thus preferably directing any bleeding from wound 10 through channel 9 to out of the patient's skin 5 at access site 12. The tissue casting 11 also typically forms within available internal bleeding pathways (not shown) in and around surrounding tissue 6, vessel 4, skin 5, and any other anatomical features (not shown) in contact with the tissue casting. All these surfaces and any instruments present typically contribute to forming the mold for creating the tissue casting. As such, the form and size of blood tissue casting 11 providing the failsafe barrier to bleeding complications as illustrated in FIG. 7, will vary significantly by patient and procedure as it molds upon different surfaces present.

Further to FIG. 7, upon removal of a sheath or other percutaneously positioned instrument (not shown), the core pin casting cavity 9, typically may be used for further access to vessel 4 for further transcoronary procedural instruments, deploying a wound closure, or the like. In the case of using compression as the wound closure approach, the core pin casting cavity 9 may simply be collapsed during compression.

Some specialized tools may be useful in carrying out the percutaneous wound closure in different ways, for example, injecting blood with syringe 2 versus ejecting blood from vessel 4 to deploy a failsafe barrier typically as described herein and in reference documents. Pulsatile indicators like those described in reference documents and elsewhere may optionally instead be used to indicate and therefore allow controlled adjustment of clotting agent pressure developing in the patient's wound site mold cavity when ensuring adequate placement to form a failsafe barrier. Radiopacity may be added to a clotting agent to enhance visualization of how well a clotting agent is deployed. Sheaths or a similar instrument, already know in the art to serve multiple useful purposes, can now also be used as a failsafe barrier mold core pin.

In some embodiments, a cannula, guidewire, needle, sheath and/or dilator used for percutaneous wound access is ported, grooved, or otherwise includes surface features modified for the purposes of procoagulation and/or vessel blood pressure pulse communication for vessel wall location (as referred to herein, a “surface-modified cannula” or “cannula”). The surface features, in some implementations, are designed to collect blood from the vessel and eject or deposit the blood along an access path to the wound site to deploy a failsafe barrier. The cannula, sheath, and/or dilator, in some examples, may range from 2 French to 30 French depending upon the style of the device (e.g., cannula, needle, guidewire, sheath, dilator, etc.). The cannula, guidewire, needle, sheath, and/or dilator, in some examples, may be formed of polymers and/or surgical grade metals.

Such a surface-modified cannula, sheath, guidewire, needle, and/or dilator, in some implementations, may be used with additional apparatus. For example, the surface-modified cannula, sheath, guidewire, needle, and/or dilator may support blood transfer and accumulation into another container, such as a syringe or a pulsatile indicator. In another example, the surface-modified cannula, sheath, guidewire, needle, and/or dilator may support the transfer of blood to or through additional clot activation apparatus or material, such as various apparatus and materials described in U.S. Pat. No. 6,159,232 to Nowakowski, incorporated by reference herein in its entirety. The materials, in some examples, may include a porous matrix such as, in some examples, glass fiber or beads, celite, kaolin, fibrin, cotton, and blood incompatible polymers or metals. In another example, the surface-modified cannula, sheath, guidewire, needle, and/or dilator may support the transfer of blood to or through additional anticoagulant neutralizing apparatus and/or agents (e.g., anticoagulant inhibitor, procoagulant, etc.), such as, in some examples, thrombin, polymers of selective electrical charge or diethylaminoethyl cellulose in catalytic form, or protamine sulfate.

In some implementations, a surface-modified cannula includes one or more surface features that are at least partially filled or obstructed with a clot activation material such that the blood is ejected after passing through the clot activation material. The clot activation material, for example, may be a procoagulant or a porous matrix. In some embodiments, obstructing the one or more surface features involves at least partially filling or obstructing the surface feature(s) with a clot activation material by inserting a device including the clot activation material into the surface-modified cannula. Conversely, in other embodiments, obstructing the one or more surface features involves inserting the cannula into a device including the clot activation material.

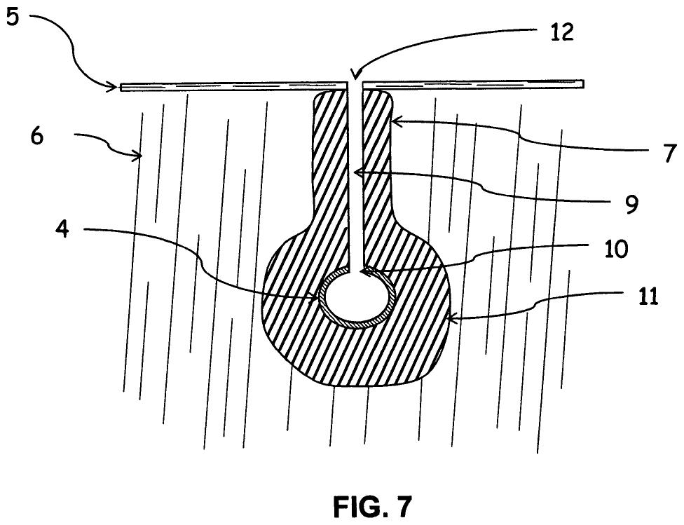

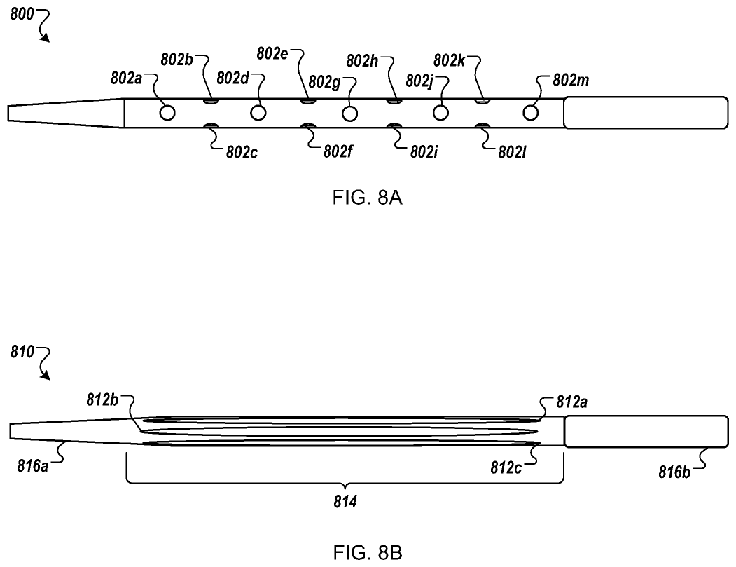

Turning to FIG. 8A, in some implementations, blood may be redirected from the vessel and ejected along an access path to the wound site using a specialized cannula 800 having at least two ports 802 including at least one distal port 802a configured to be positioned proximate to an exterior wall of the vascular wound and at least one proximal port 802m. In operation, blood may be received by one or more distal ports 802 including the distal port 802a and ejected along the access path to the wound site via additional ports, such as ports 802b through 802m. Further, blood may be received by an opening in the distal end of the specialized cannula 800 and ejected via the ports 802. Beyond the visible ports 802a through 802m, the specialized cannula 800 may include additional ports, such as five ports disposed generally opposite ports 802a, 802d, 802g 802j, and 802m. For example, blood may be collected by at least two ports including distal port 802a and another distal port disposed approximately opposite to distal port 802a.

As illustrated, the ports 802 may be generally circular in shape. In other embodiments, at least a portion of the ports 802 may be elliptical, tear drop shaped, or elongated slots. The ports 802, in some examples, may be arranged annularly, axially, or serpentine about the surface. In some embodiments, the ports form an open spring similar to a coil about the surface of the hollow shaft. For example, a portion of the hollow shaft including the ports 802 may be formed as a spring segment having gaps between the coils. The flexible spring segment may be particularly useful in embodiments configured as a guidewire style cannula. Alternatively, materials having spring like properties may be configured axially, and compressed annularly to advance through a cannula. Upon exiting the distal end of a cannula, they expand radially, so when partially withdrawn from a blood vessel, they tent open the vessel wound thus allowing blood to eject into the tissue tract.

Turning to FIG. 8B, in some implementations, blood may be redirected from the wound site in the vessel and ejected along an access path to the wound site using a specialized cannula 810 having a distal end 816a, a proximal end 816b, and at least one channel 812 creating a depression in a surface of a shaft region 814 of the cannula 810. As illustrated, the cannula 810 includes at least three channels 812a, 812b, and 812c, each extending in parallel along a significant portion of a length of the shaft 814. In some embodiments, the cannula 810 may include two or three additional channels 812 disposed on an opposite side (not visible in FIG. 8B) of the shaft 814 of the channels 812a to 812c. In operation, blood from proximate the wound site may be received in each channel 812 at a point closest to the distal end 816a of the specialized cannula 810 and ejected along the access path to the wound site as the blood flows along each of the channels 812.

As illustrated the channels 812 are substantially identical in length and arranged in parallel on the shaft 814. The channels 812, for example, may be evenly disposed around a circumference of the shaft 814. In other embodiments, one or more channels may curve around the circumference of the shaft 814 and/or follow a zig-zag or sinusoidal pattern. In further embodiments, channels of varying lengths may be arranged along the shaft of a surface-modified cannula. In some embodiments, the channels form a flexible bellows about the surface of the hollow shaft. For example, a portion of the hollow shaft including the channels 812 may be formed as an annular or spiral bellows segment having ridges and depressions.

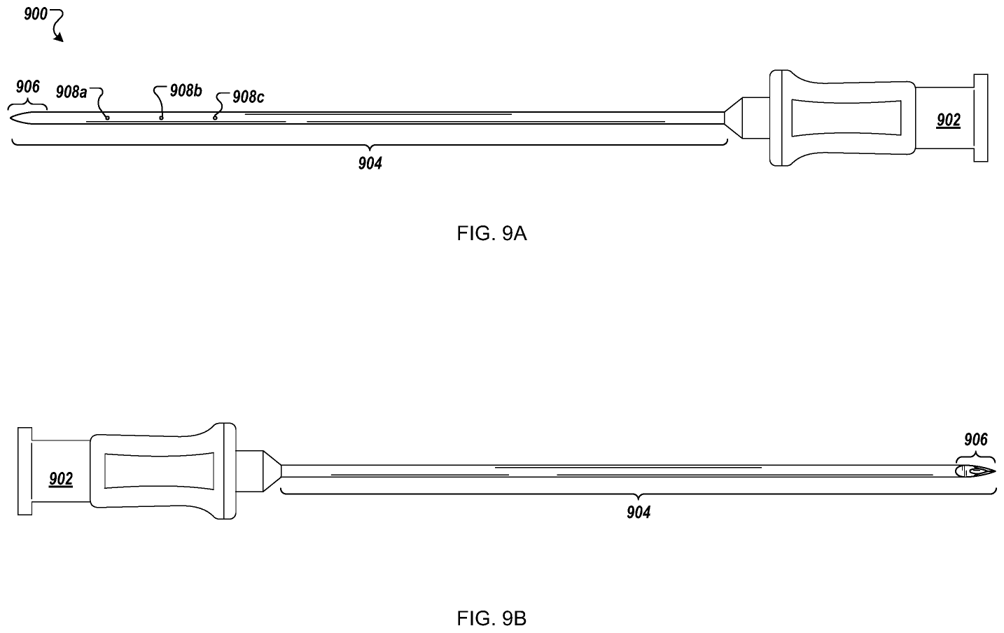

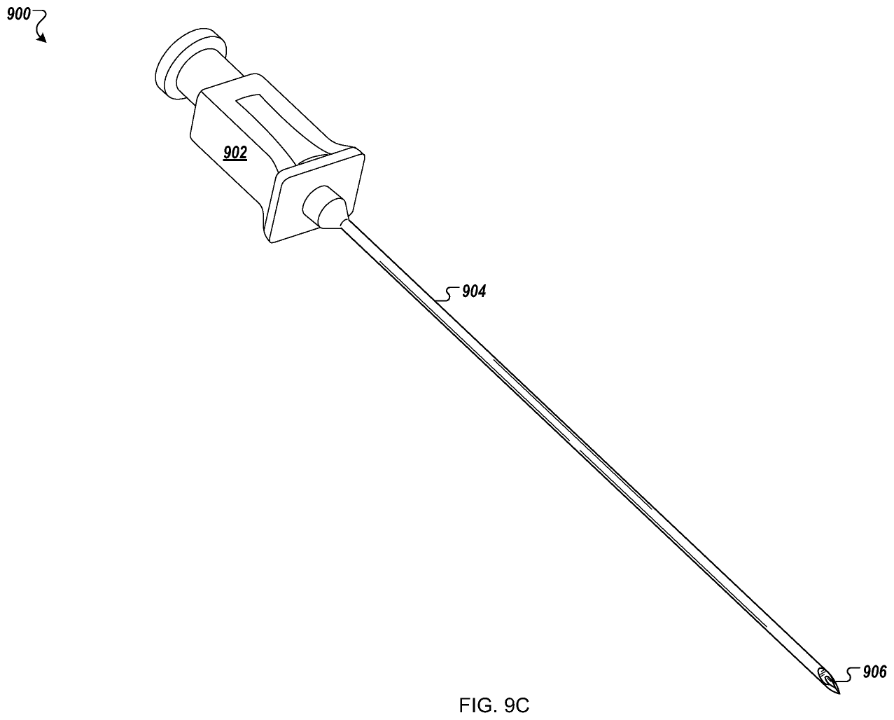

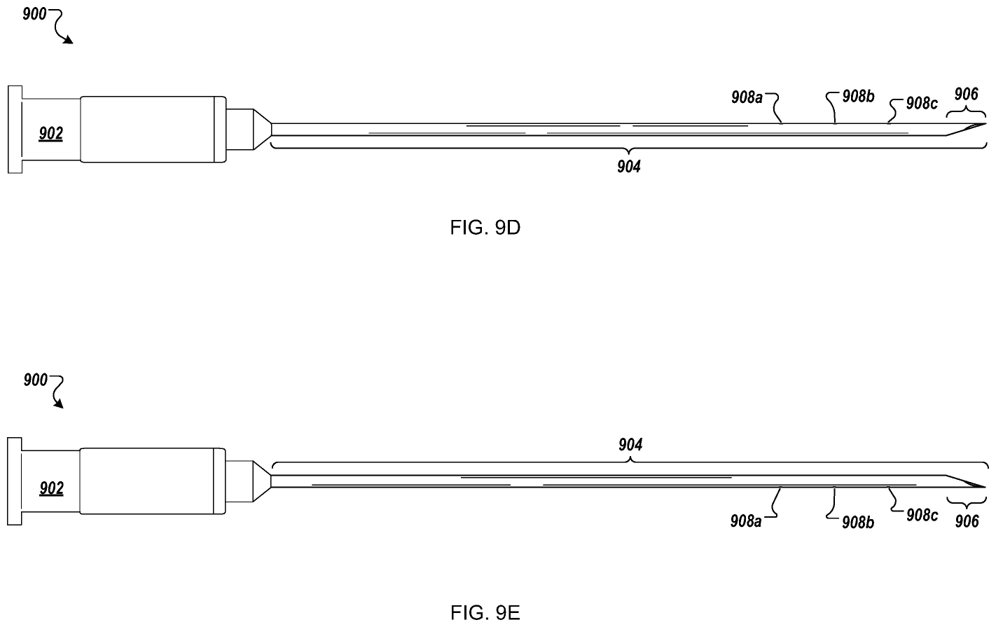

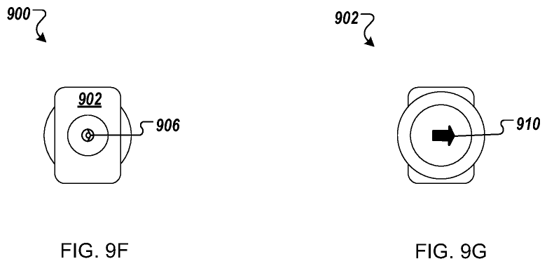

Turning to FIGS. 9A through 9G, a surface-modified needle or cannula 900 (e.g., a Seldinger style needle) includes a handle or hub 902, a hollow shaft 904 including a bevel portion 906, and a series of ports 908a-c. As discerned by comparing FIG. 9A to FIG. 9B, the ports 908a-c are preferably aligned opposite a bevel cut (e.g., on the longest sharp side of the hollow shaft 904). As illustrated in viewing all sides of the hollow shaft (e.g., including side views of FIG. 9C through FIG. 9E), only one side includes ports 908. In other embodiments, ports may be arranged on multiple sides of the hollow shaft 904.

As illustrated in FIG. 9G, in a top view of the handle or hub 902, an orientation marking 910 points in an orientation of the bevel 906 of the hollow shaft 904. The orientation marking 910, for example, may also indicate an orientation of the ports 908a-c on the opposing side of the hollow shaft 904. In other embodiments, rather than or in addition to an indicator marked (e.g., with ink or etching) in a top surface of the hub or handle 902, an orientation marking may be positioned on a side of the hub or handle 902. In further examples, a shape of the hub or handle 902 may be modified to create a visual and tactile indication of the orientation of the bevel 906/ports 908.

In some implementations, surface-modified cannulas are integrated with bleeding pathway management methods within existing apparatus used for accessing a blood vessel. Although described above in a preferred embodiment in relation to Seldinger apparatus modification, the novel teachings provided herein may be applied to a wide variety of other vessel access procedures without limitation, such as dialysis procedures and other procedures that use a needle/cannula to access a blood vessel. The wide variety of applications can also provide a variety of different beneficial purpose outcomes. By example, percutaneous Seldinger access procedures have different benefits associated with different points of access. In illustration, femoral artery access also includes protection against retroperitoneal internal bleeding, while such potential bleeding in radial artery access does not exist primarily due to isolating anatomical distance. In contrast, other benefits such as reducing risks associated with hematoma remain common to both procedures.

Generally, in illustrative embodiments, instruments used for vascular access can be modified to provide new added functions including conveyance of a sealing material such as clotting blood, while the instruments retain their pre-existing functions. These functions, for example, typically include accessing a blood vessel, dilating a blood vessel, and providing intravascular conveyance of fluids and/or other medical instruments. For illustrative purposes, a common Seldinger needle may be optimized to convey clotting blood to anatomical spaces about an arteriotomy. Other instrumentation comprising a dilator, guidewire, sheath, catheter, and such may likewise be modified with the novel teachings provided. Generally, with the needle or cannula placed within a blood vessel, blood may be conveyed either along the outside surface of the needle/cannula and/or through the needle/cannula lumen. To convey blood along the outside of the needle/cannula, in one example, a variable outer diameter may be applied such as an axial fluting of the needle/cannula surface. Such surface variation, for example, can allow blood to escape the accessed vessel due to the lack of a perfect seal between the vessel wall tissue and the full circumference of the needle/cannula surface. In a preferred example of needle/cannula modification, the needle shaft may be ported with one or more holes allowing transfer of a sealing material such as clotting blood from within the needle lumen to the anatomical environment surrounding the needle. This second type of apparatus modification, for example, provides the benefits of simplicity of design and precision it enables.

A Seldinger needle may be ported with one or more holes of such size and location to provide the newly added function of clotting blood placement about an arteriotomy. Hole sizing and placement are preferably selected to optimize flow of clotting blood about the wound area while retaining beneficial timing of such placement to be simply and fully integrated within the preferred established steps of the surgical procedure.

Holes sizing and number of holes can vary widely, and teaching provided herein can be readily optimized for a wide variety of medical applications by those skilled in the art. Staying with blood as the illustrative example, blood cells typically range from 6 to 8 microns in size and readily flow without difficulty through 40-micron filter sized extracorporeal circuits. Commonly available medical grade hypotubing inner diameters that may provide blood flow to the one or more holes range from a nominal 0.008 inches in diameter for a 30-gage needle to 0.173 inches in diameter for a 6-gage needle. Compression of tissue covering the needle surface hole or holes and back pressure from other fluids already present outside the hole or holes in a particular application can also impact flow rate, as can variability in physiological conditions from patient to patient and the user defined optimal dwell time desired to dispense a target volume for a particular need. Mathematical modeling using finite element analysis and related tools known in the engineering arts can help target design choices and be refined as needed through clinical trial of the medical application of interest.

### Clinical Example

An 18-gauge Seldinger needle shaft 2¾ inches in length was ported with three holes each 0.015 inches in diameter. A femtosecond laser pulse used to make the holes allows manufacturing repetitive accuracy measured in microns, so hole size can easily be further optimized as preferred. In the present example, clinical use in the presence of 115 beats per minute (bpm) heart rate with 78 mmHg blood pressure and 147 ACT (activated clotting time) provided a flow rate of 0.82 mL per second, or a 12 second duration to achieve an arbitrary volume placement of 10 mL dispersed about the vessel access site and within internal bleeding pathways. After 12 seconds, the interventionalist practitioner inserted a guidewire through the needle, thus acting as a valve shutoff to further blood placement, and as is normally performed in the next step of standard Seldinger access procedure. Thus, the new apparatus function combination provided for traditional Seldinger access with an unhurried 12 seconds added to the procedure that may save a patient's life from internal bleeding.

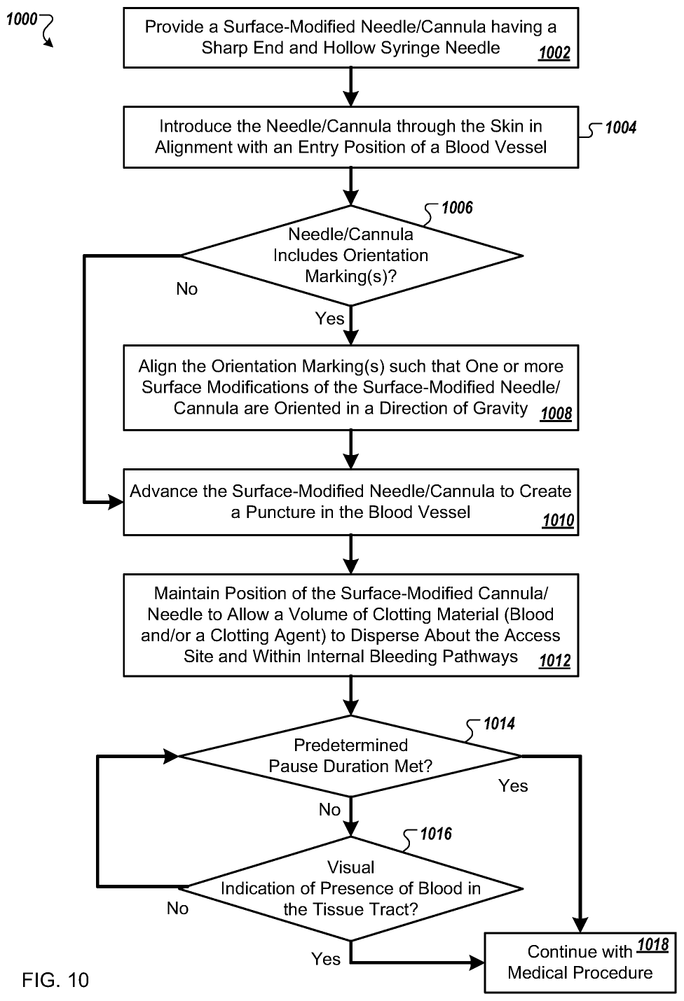

Turning to FIG. 10, a flow chart illustrates an example method 1000 for using a surface-modified needle or cannula in a medical procedure. The method 1000, for example, may be performed using any of the surface-modified cannulas of FIG. 8A, FIG. 8B, and/or FIG. 9A through 9G.

In some implementations, the method 1000 begins with providing a surface-modified needle or cannula having a sharp end and hollow syringe needle (1002). The surface-modified needle or cannula, for example, may be packaged in a kit with other apparatus for forming the medical procedure. A shaft of the surface-modified needle or cannula may be flexible, semi-flexible, or rigid, depending upon the design and use. In a preferred embodiment, the surface modified needle or cannula includes a rigid hollow shaft for performing a modified Seldinger process. The surface-modified needle or cannula may include surface modifications such as, in some examples, one or more channels, openings (ports), and/or other differentiations in needle diameter (e.g., fluting) allowing for blood flow between a surface of the needle or cannula and a blood vessel lumen, blood vessel wall, and access tissue tract.

In some implementations, the needle or cannula is introduced through the skin of a patient in alignment with an entry position of a blood vessel (1004). The procedure, for example, may be a Seldinger process involving introduction of medical tools into a blood vessel such as an artery.

In some implementations, if the surface-modified needle or cannula includes one or more orientation markings (1006), the orientation marking(s) are aligned by the practitioner such that one or more surface modifications are preferentially oriented in a direction of gravity (1008). The orientation, for example, may assist in movement of blood through one or more ports of the surface-modified needle or cannula and direct any external blood flow toward the patient skin surface and not upward toward the practitioner. The orientation markings, for example, may include a bevel indicator marked on a surface of a handle of the surface-modified needle or cannula indicating an orientation of a bevel (e.g., needle edge) of the surface-modified needle or cannula, where the bevel is aligned with at least a portion of the surface modification(s). In another example, the orientation marking(s) may include a surface modification (e.g., dimple, raised arrow, etc.) of the handle that visually and tactically indicates an orientation of the bevel and/or at least a portion of the surface modification(s). In a further example, a shape of a top of the handle of the surface-modified cannula or needle may indicate the orientation of the bevel and/or at least a portion of the surface modification(s).

In some implementations, the surface-modified needle/cannula is advanced by the practitioner to create a puncture in the blood vessel (1010). The surface-modified needle/cannula may include a modified (e.g., dimpled) surface treatment near the bevel of the needle or cannula to improve imaging to discern position of the bevel during advancement to create the puncture.

In some implementations, a position of the surface-modified cannula or needle is maintained to allow a volume of clotting material (e.g., blood and/or a clotting agent) to disperse about the access site and within internal bleeding pathways (1012).

In some embodiments, as blood escapes the wound site in the blood vessel via the surface-modified needle or cannula, a user maintains the surface-modified needle or cannula in place for a predetermined pause duration (1014), such as between one second and three minutes. The predetermined period of time, in some preferred embodiments, may include at least 2 seconds, at least 10 seconds, at least 15 seconds, or about 30 seconds, although time will vary depending upon the diameter of the needle or cannula as well as the design and/or distribution of the surface features. Once the predetermined pause duration has passed, the practitioner may continue with the medical procedure (1018). For example, the practitioner may replace the needle or cannula with another medical device such as a sheath, needle, catheter, and/or probe.

In some implementations, if a visual indication of presence of blood in the tissue tract is identified (1016), the practitioner may continue with the medical procedure (1018). Instead of or in addition to the predetermined period of time, in some embodiments, the practitioner watches for a visual indication of presence of blood in the tissue tract, such as swelling at the surface of the skin and/or blood escaping from the skin level.

During the medical procedure, the blood redirected to the region around the wound site and in the tissue tract along the access path by the surface-modified cannula will clot around vessel, along the tissue tract, and within internal bleeding pathways subject to individual patient anatomy variation for the duration of the medical procedure, thus sealing off internal bleeding pathways. While such a medical procedure may typically conclude with a twenty-minute manual compression period and a topical bandage, due to the sealing process occurring during the medical procedure, standard manual compression may not be required. Further, no closure device may be required, and there may be no delay in patient ambulation.

Although referenced in the method 1000 as a surface-modified needle or cannula, in additional examples, the device may be sheath, guidewire, needle, and/or dilator, depending upon the medical procedure.

Some embodiments may also include kits including or consisting of any devices or combinations of devices described herein or though related references, and typically instructions for their use. Examples of devices suitable for kits include a Seldinger needle, a dilator, a sheath, a guidewire, a catheter, a cannula, a surface-modified cannula, a blood dispensing tool, a syringe, and/or a pressure gauge. A comprehensive kit may preferably include components required to perform intravascular access such as through Seldinger technique, components useful in forming a failsafe barrier to bleeding, components useful in performing a wound closure, and instructions for use. The instructions for use, for example, may instruct a practitioner to perform steps as discussed in relation to various methods and apparatus described herein. As illustrative example, instructions may direct how a guidewire style cannula should extend through a needle into the vessel and the encasing needle cannula then removed as in traditional Seldinger technique, but upon removal of the needle from the patient, there is pause until evidence of blood flow though the tissue tract exiting at the skin surface, before advancing a traditional Seldinger dilator-sheath assembly over the guidewire into the vessel. Having labels or having instructions for use may be separate or in any combination with a kit and typically provided by a manufacturer, a seller, or a distributor of any form of kit, and done so in any manner allowed by a governing agency, such as the United States Food and Drug Administration. Any and all kit examples above may be recombined, added to, or deleted from, as may be the preference.

All patents, patent publications, and peer-reviewed publications (i.e., “references”) cited as part of the present patent application are expressly incorporated by reference to the same extent as if each individual reference were specifically and individually indicated as being incorporated by reference. In case of conflict between the present disclosure and the incorporated references, the present disclosure controls.

It is understood that the invention is not confined to the particular construction and arrangement of parts herein illustrated and described, but embraces such modified forms thereof as come within the scope of the claims.

What is claimed is:

1. A kit for use in forming a failsafe percutaneous wound barrier, the kit comprising:
a surface-modified cannula comprising
a hollow shaft comprising
a distal end,
a proximal end comprising an opening, and
at least one port in the hollow shaft between the distal end and the proximal end; and
instructions for forming the failsafe percutaneous wound barrier, the instructions directing a practitioner to
advance the surface-modified cannula along an access path through skin of a patient to a position proximate or in a wound in a blood vessel, and
prior to performing another medical procedure, maintain the position of the cannula within the access path for a predetermined period of time or until viewing physical evidence of blood present in the tissue tract, thereby enabling the at least one port to disperse blood from the wound along the access path;
wherein the blood, after being dispersed along the access path, at least partially solidifies into a tissue mass over a second period of time during which a medical procedure is conducted via the access path.

2. The kit of claim 1, wherein the predetermined period of time is at least three seconds.

3. The kit of claim 1, wherein the instructions form a modified version of the Seldinger procedure.

4. The kit of claim 1, wherein, upon removing the cannula or other access device from the access path after performing the medical procedure, a core pin channel impression in the form of a portion of the surface-modified cannula is exposed via an opening in the skin of the patient, wherein
the core pin channel impression provides a channel for directing any bleeding out to the skin of the patient, thereby providing a failsafe mechanism to avoid internal bleeding.

5. The kit of claim 4, wherein the instructions comprise directing the practitioner to perform post procedure wound management with the established core pin channel.

6. The kit of claim 1, wherein the at least one port is round.

7. The kit of claim 1, wherein the distal end comprises a sharp bezel.

8. The kit of claim 7, wherein the at least one port is aligned with a point of the bezel.

9. The kit of claim 1, wherein the distal end comprises a hub.

10. The kit of claim 1, wherein the hub comprises an orientation marker referencing an orientation of the at least one port.

11. The kit of claim 10, wherein the orientation marker is a tactile orientation marker.

12. The kit of claim 10, wherein the instructions comprise directing the practitioner to orient the at least one port in a downward position, thereby directing blood flow away from the practitioner while one or more ports of the at least one port is outside the patient.

13. The kit of claim 1, wherein the at least one port comprises three ports aligned along a portion of a length of the hollow shaft of the surface-modified cannula.

14. The kit of claim 1, wherein the hollow shaft is rigid.

15. A kit for use in forming a failsafe percutaneous wound barrier, the kit comprising:
a surface-modified cannula comprising
a hollow shaft comprising an inner diameter, an outer diameter, and an opening, and
one or more surface features along a length of the hollow shaft, the one or more surface features comprising one or more depressions, and/or one or more ports, each surface feature extending at least partially between the outer diameter and the inner diameter of the hollow shaft; and
instructions for forming the failsafe percutaneous wound barrier, the instructions comprising instructions directing a practitioner to
position the surface-modified cannula at or in a blood vessel via an access path through a tissue tract of a patient by performing a Seldinger technique, and
maintain the position of the surface-modified cannula within the tissue tract for a predetermined period of time or until viewing physical evidence of blood present in the tissue tract prior to proceeding with a medical procedure, thereby enabling the blood to migrate, via the one or more surface features, from a blood vessel opening to a region surrounding the blood vessel opening and along the tissue tract;
wherein the blood, after migrating to the region, at least partially solidifies into a tissue mass over a second period of time during which a medical procedure is conducted via the access path.

16. The kit of claim 15, wherein, upon removing the surface-modified cannula or other access device from the tissue tract after performing the medical procedure, a core pin channel impression in the form of a portion of the surface-modified cannula is exposed via an opening in the skin of the patient, wherein
the core pin channel impression provides a channel for directing any bleeding out to the skin of the patient, thereby providing a failsafe mechanism to avoid internal bleeding.

17. The kit of claim 15, wherein the at least one port comprises at least one distally-positioned port proximate the distal opening, and at least one additional port spaced along a length of the hollow shaft from the distally-positioned port.

18. The kit of claim 17, wherein, when the surface-modified cannula is positioned at or in the blood vessel, the at least one proximally-positioned port is disposed in the tissue tract.

19. The kit of claim 15, at least one surface feature of the one or more surface features is configured to enable, when the surface-modified cannula is positioned at or in the blood vessel, direction of a portion of the blood to exterior to the surface of the skin of the patient.

20. The kit of claim 15, wherein the predetermined period of time is between 3 seconds and 1 minute.
